(19) United States
(12) Patent Application Publication
(10) Pub. No.: US 2023/0416979 A1
(43) Pub. Date: Dec. 28, 2023

(54) SILK-BASED ARTICLES HAVING DECREASED WATER UPTAKE AND IMPROVED MECHANICAL PROPERTIES, AND METHODS OF MAKING AND USING THE SAME

(71) Applicant: Trustees of Tufts College, Medford, MA (US)

(72) Inventors: Junqi Wu, Medford, MA (US); David L. Kaplan, Concord, MA (US); Vincent P. Fitzpatrick, Somerville, MA (US)

(21) Appl. No.: 18/342,271

(22) Filed: Jun. 27, 2023

Related U.S. Application Data
(63) Continuation of application No. PCT/US2021/073206, filed on Dec. 31, 2021.
(60) Provisional application No. 63/202,572, filed on Jun. 16, 2021.
(60) Provisional application No. 63/132,976, filed on Dec. 31, 2020.

(51) Int. Cl. D06M 15/15; D06M 16/00

(52) U.S. Cl. CPC D06M 15/15; D06M 16/003

(57) ABSTRACT

Coated silk fibroin articles and methods of making and using the same are disclosed. The articles have a reduced water uptake and improved mechanical properties when compared with uncoated comparable articles. The article includes a silk fibroin article core and a biodegradable hydrophobic polymer layer substantially encompassing the silk fibroin article core. The article has either: (i) at least one mechanical property that is at least 50% greater than a reference mechanical property of the silk fibroin article core in the absence of the biodegradable hydrophobic polymer layer, wherein the at least one mechanical property includes a three-point bending flexural strain; or (ii) a water uptake that is at least 50% lower than a reference water uptake of the silk fibroin article core in the absence of the biodegradable hydrophobic polymer layer.

### CROSS-REFERENCE TO RELATED APPLICATIONS

This application is a bypass continuation of International Application Serial No. PCT/US2021/073206, filed Dec. 31, 2021 (2095.0397). International Application Serial No. PCT/US2021/073206 is related to, claims priority to, and incorporates by reference herein for all purposes U.S. Provisional Patent Application No. 63/132,976, filed Dec. 31, 2020 (2095.0394), and U.S. Provisional Patent Application No. 63/202,572, filed Jun. 16, 2021 (2095.0396). Each of the foregoing patent applications is incorporated herein by reference in their entirety for all purposes.

### STATEMENT REGARDING FEDERALLY FUNDED RESEARCH

This invention was made with government support under grants EB027062 awarded by the National Institutes of Health and FA9550-17-1-0333 awarded by the United States Air Force. The government has certain rights in the invention.

### BACKGROUND

Silk materials that are processed from amorphous bulk materials using thermal processing and/or high pressure processing were invented recently and overcame a variety of technical hurdles in the silk fibroin material space, including but not limited to, providing improved machinability and other mechanical features that are relevant to use in the medical field. However, several technical challenges remain with these materials, including reducing their water uptake and maintaining mechanical properties over time in a biological environment.

In an entirely different part of the materials space, polylactide (PLA) or polycaprolactone (PCL) materials possess many of the desirable material properties that are desirable for use in the medical space, but these materials suffer from their own disadvantages. For example, use of these materials in large quantities is believed to cause inflammation due to localized pH reductions that result from their dissolution. As a result, their use in biomedical applications has been limited.

A material is needed for use in biomedical applications that has superior mechanical properties while overcoming the technical shortcomings of the existing options described above. In addition, a broader need exists for biomaterials having controllable degradation properties.

### SUMMARY

In an aspect, the present disclosure provides a coated silk fibroin article. The coated silk fibroin article has a reduced water uptake and improved mechanical properties. The coated silk fibroin article has a silk fibroin article core and a biodegradeable hydrophobic polymer layer substantially encompassing the silk fibroin article core. The coated silk fibroin article has either: (i) at least one mechanical property that is at least 50% greater than a reference mechanical property of the silk fibroin article core in the absence of the biodegradable hydrophobic polymer layer, wherein the at least one mechanical property includes a three-point bending flexural strain; or (ii) a water uptake that is at least 50% lower than a reference water uptake of the silk fibroin article core in the absence of the biodegradable hydrophobic polymer layer.

In another aspect, the present disclosure provides a method. The method includes: (i) selecting an elevated temperature and/or an elevated pressure to produce a desired silk fibroin article core of a desired crystallinity and desired material properties; (ii) selecting a biodegradable polymer and a desired coating thickness to produce a desired coated silk fibroin article having at least one desired mechanical property or at least one desired water uptake, the selecting of step (ii) taking the desired crystallinity and the desired material properties into account; (iii) applying the elevated temperature and/or the elevated pressure to a silk fibroin material comprising substantially amorphous structure to form a silk fibroin article core, wherein the silk fibroin article core has the desired crystallinity and desired material properties; and (iv) coating the silk fibroin article core with a biodegradable hydrophobic polymer layer comprising the biodegradable polymer and having the desired coating thickness to form a coated silk fibroin article.

In a further aspect, the present disclosure provides a method. The method includes: (i) providing a silk fibroin material having substantially amorphous structure; (ii) applying at least one of elevated temperature and elevated pressure to the silk fibroin material to form a silk fibroin article core, wherein the applying induces fusion between at least a portion of the silk fibroin and structural change of fibroin in the silk fibroin material; and (iii) coating the silk fibroin article core with a biodegradable hydrophobic polymer layer to form a coated silk fibroin article.

In yet another aspect, the present disclosure provides a method. The method includes: (i) selecting an elevated temperature and/or an elevated pressure to produce a desired silk fibroin article core of a desired crystallinity and desired material properties; (ii) applying the elevated temperature and/or the elevated pressure to a silk fibroin material comprising substantially amorphous structure to form a silk fibroin article core, wherein the silk fibroin article core has the desired crystallinity and the desired material properties; and (iii) coating the silk fibroin article core with a biodegradable hydrophobic polymer layer to form a coated silk fibroin article.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTIONS OF THE DRAWINGS

FIG. 1 is a plot of water uptake measurements, as described in Example 1.

FIG. 2 is a plot of flexural strain measurements, as described in Example 1.

### DETAILED DESCRIPTION

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biocompatible: the term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: as used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Compaction: as used herein, the term “compaction” refers to a process by which a material progressively loses its porosity due to the effects of loading.

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Fusion: as used herein, the term “fusion” refers to a process of combining two or more distinct entities into a new whole.

Hydrophilic: as used herein, the term “hydrophilic” and/or “polar” refers to a tendency to mix with, or dissolve easily in, water.

Hydrophobic: as used herein, the term “hydrophobic” and/or “non-polar”, refers to a tendency to repel, not combine with, or an inability to dissolve easily in, water.

Improve, increase, or reduce: as used herein or grammatical equivalents thereof, indicate values that are relative to a baseline measurement, such as a measurement in a similar composition made according to previously known methods.

Macroparticle: as used herein, the term “macroparticle” refers to a particle having a diameter of at least 1 millimeter. In some embodiments, macroparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of macroparticles if the mean diameter of the population is equal to or greater than 1 millimeter.

Microparticle: as used herein, the term “microparticle” refers to a particle having a diameter between 1 micrometer and 1 millimeter. In some embodiments, microparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of microparticles if the mean diameter of the population is between 1 micrometer and 1 millimeter.

Nanoparticle: as used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of nanoparticles if the mean diameter of the population is equal to or less than 1000 nm.

Physiological conditions: as used herein, has its art-understood meaning referencing conditions under which cells or organisms live and/or reproduce. In some embodiments, the term refers to conditions of the external or internal mileu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Pure: as used herein, a material, additive, and/or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: as used herein describes a standard or control relative to which a comparison is performed. For example, in some embodiments, a material, article, additive, entity or other sample, sequence or value of interest is compared with a reference or control material, article, additive, entity or other sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Solid form: as is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized as a single such form (e.g., as a pure preparation of a single polymorph). In some embodiments, such entities may be utilized as a mixture of such forms.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Compositions and Methods

The present disclosure provides coated silk fibroin articles and methods of making and using the same.

A method of making a coated silk fibroin article includes: (i) selecting an elevated temperature and/or an elevated pressure to produce a desired silk fibroin article core of a desired crystallinity and desired material properties; (ii) selecting a biodegradable polymer and a desired coating thickness to produce a desired coated silk fibroin article having at least one desired mechanical property or at least one desired water uptake, the selecting of step (ii) taking the desired crystallinity and the desired material properties into account; (iii) applying the elevated temperature and/or the elevated pressure to a silk fibroin material comprising substantially amorphous structure to form a silk fibroin article core, wherein the silk fibroin article core has the desired crystallinity and desired material properties; and (iv) coating the silk fibroin article core with a biodegradable hydrophobic polymer layer comprising the biodegradable polymer and having the desired coating thickness to form a coated silk fibroin article.

A method of making a coated silk fibroin article includes: (i) providing a silk fibroin material comprising substantially amorphous structure; (ii) applying at least one of elevated temperature and elevated pressure to the silk fibroin material to form a silk fibroin article core, wherein the applying induces fusion between at least a portion of the silk fibroin and structural change of fibroin in the silk fibroin material; and (iii) coating the silk fibroin article core with a biodegradable hydrophobic polymer layer to form a coated silk fibroin article.

A method of making a coated silk fibroin article includes: (i) selecting an elevated temperature and/or an elevated pressure to produce a desired silk fibroin article core of a desired crystallinity and desired material properties; (ii) applying the elevated temperature and/or the elevated pressure to a silk fibroin material comprising substantially amorphous structure to form a silk fibroin article core, wherein the silk fibroin article core has the desired crystallinity and the desired material properties; and (iii) coating the silk fibroin article core with a biodegradable hydrophobic polymer layer to form a coated silk fibroin article. The applying of step (ii) can induce fusion between at least a portion of the silk fibroin and structural change of fibroin in the silk fibroin material.

The coating step of any of the methods described herein can include three-dimensional printing, micro-scale mixing, spraying, dip coating, thermal molding (in concert with or in addition to the thermal molding that forms the core of the silk article), or a combination thereof. In reference to one specific, non-limiting example, relating to three-dimensional printing methods, the coating can be designed using software known to those having ordinary skill in the art (for example, CAD software, available from Solidworks, Waltham, MA), taking into account the dimensions of the selected silk articles. The thickness of the 3D-printed coating can be tuned using the software. In another specific, non-limiting example, relating to the case of micro-scale mixing, a powder of the coating material can be mixed with silk amorphous nanoparticles via analytical milling (20,000 rpm for 2 minutes using a Cole-Parmer (headquarters Vernon Hills, IL) analytical mill) and thermal molding. The different mixing ratio can affect the hydrophobicity of the final coated silk article. In yet another specific, non-limiting example, relating to the case of dip-coating, organic solvents in which silk is insoluble are used. The thickness of the coating is dependent on the concentration of the coating material, the number of layers, and the thickness of individual layers. The organic solvent can be removed through drying in a fumehood after the coating process. In the case of thermal spray coating, the thickness of the coating can depend on coating speed and spray layer thickness. In some cases, thermal molding itself can be used to form the coatings around preformed silk article cores. As an alternative, thermal molding could be used to form shells of the coating material, followed by filling the shells with silk and thermomolding to form the coated article.

In some embodiments, methods disclosed herein involve the fabrication of amorphous silk nanomaterials (ASN) generated from aqueous silk fibroin solution. ASN may then be treated by hot pressing, leading to fusion and densification of the silk (e.g., into a silk article core). The resulting silk bulk material exhibits specific strength higher than that of most natural structural materials and has been shown effective for fabricating silk-based composites. In addition, it is shown that the engineered silk material has thermoforming properties, which allows the materials to be further transformed to desirable shapes under proper conditions. In some embodiments, compositions and methods described herein demonstrate a thermal and pressure-based, time-efficient and controllable method to transform silk fibroin from a silk fibroin material including substantial amounts of amorphous silk fibroin (for example, in powder form) directly to bulk structural material, which can function as the cores described herein. In some embodiments, methods and compositions described herein may allow for the application of more traditional process and molding techniques to silk materials, where this was not previously successfully employed for silk. Additionally, in some embodiments, processing methods described herein avoid the need for solvent or aqueous approaches, and providing direct routes to transform silk fibroin material into part cores. In accordance with various embodiments, methods described herein provide for the transformation of silk fibroin from amorphous materials to a semi-crystalline high-performance structural material through controlled application of heat and pressure. In some embodiments, provided processes induce a conformation transition of silk molecules from random coil to β-sheet. In some embodiments, provided methods include the processing of natural silk fiber into amorphous silk material (e.g., powder) via degumming, silk fibroin solubilization and freeze drying to prepare the proper premolding materials; feeding the amorphous silk material into a predesigned mold; and inducing the conformation and structure change of silk by applying heat and pressure. Additionally, this method can be processed with silk alone, or with the addition of inorganic fillers or second polymers to generate composite devices.

In some cases, the methods described herein can include selecting an elevated temperature and an elevated pressure to produce a desired silk fibroin article core of a desired crystallinity and desired material properties and then applying that elevated temperature and elevated pressure to a silk fibroin material having substantially amorphous structure. That is, the methods described herein can predictably select and apply temperatures and pressures to produce article cores having desired crystallinity and material properties.

Coated Silk Fibroin Articles

The coated silk fibroin articles described herein include a silk fibroin article core and a biodegradable hydrophobic polymer layer substantially encompassing the silk fibroin article core. The silk fibroin article core can be a thermally-processed silk fibroin article core or a pressure-processed silk fibroin article core, as described herein.

The present disclosure includes multiple related technical advances with respect to the production of silk fibroin materials that have improved mechanical properties. The general idea of coating silk fibroin materials has been previously contemplated, including efforts to enhance hydrophobicity with methods including one-step atomic layer deposition of TiO₂, plasma-polymerized hexamethyldisiloxane (HMDSO) deposition, and surface modification with nano-SiO₂. The inventors discovered that a biodegradable hydrophobic polymer coating (i.e., a specific class and quality of coating) that is applied to a thermally-processed silk fibroin articles (i.e., a specific type of article that is made from a specific material) provides unexpectedly large improvements in multiple performance characteristics that are relevant to the use of thermally-processed silk fibroin articles as mechanically-strong biomaterials. Two prominent improvements are an impressive reduction in the water uptake of articles and the mechanical properties as measured by a three-point bending test. The reduction in water uptake is unexpectedly improved relative to other approaches that the inventors tried before the inventive combination disclosed herein. The improved mechanical properties are unexpectedly large in scale. The uncoated thermally-processed silk fibroin article has mechanical properties that are inferior to polylactide, a conventional biodegradable hydrophobic polymer that has shortcomings that are described elsewhere herein, having similar size and shape. Specifically, the flexural strain (as measured in a three-point bending test) that can be achieved by polylactide, and other conventional biodegradable hydrophobic polymers, is orders of magnitude greater than can be achieved by an uncoated thermally-processed silk article. However, the coated silk fibroin article disclosed herein has mechanical properties that are compatable to polylactide.

Thermally- and pressure-processed silk articles can have limited utility in applications that have strict mechanical property requirements, due at least in part to the uptake of water, which can compromise the mechanical properties in aqueous environments. Uncoated silk fibroin bone screws made by the methods described herein for making silk fibroin article cores have been reported to absorb up to 35% by weight of water when immersed in a PBS solution at 37° C. for 8 hours. This water uptake results in swelling of the articles and reduction in some of the mechanical properties, including stiffness. The disclosed articles and methods overcome some of these shortcomings.

The biodegradable hydrophobic polymer layer can comprise aliphatic polymers, such as polylactide, polycaprolactone, and polycarbonate, aromatic and aliphatic polyanhydrides, polyurethanes, polyamides, poly(ester amide), or a copolymer or combination thereof. In some cases, the biodegradable hydrophobic polymer layer includes polylactide or polycaprolactone.

In some cases, the biodegradable hydrophobic polymer layer can have a substantially uniform thickness. The average thickness of the biodegradable hydrophobic polymer layer can be between 1 m and 5 mm.

The biodegradable hydrophobic polymer layer can account for no more than 50%, no more than 25%, no more than 15%, or no more than 10% by weight of the coated silk fibroin article. The biodegradable hydrophobic polymer layer can account for at least 1% by weight of the coated silk fibroin article. The specific weight of the biodegradable hydrophobic polymer layer can be dependent on the chosen coating method or the desired mechanical properties.

The biodegradable hydrophobic polymer layer can comprise an acid-activated protease. The acid-activated protease can be selected from the group consisting of pepsin, a cathepsin, renin, and combinations thereof. The acid-activated protease is triggered by a local pH reduction initiated by dissolution or degradation of the biodegradable hydrophobic polymer layer. It should be appreciated that this local pH reduction is typically considered a shortcoming of the materials included in the biodegradable hydrophobic polymer layer, because of the biological inflammation response that it triggers. But in this case, the local pH reduction is used as a trigger to initiate the degradation of the material itself. The acid-activated protease can be present in the biodegradable hydrophobic polymer layer in an amount by weight of between 0.1 wt % and 5.0 wt %, including but not limited to, between 0.5 wt % and 4.0 wt %, or between 1.0 wt % and 3.0 wt %.

Silk Materials

Any of a variety of silk materials may be used in accordance with various embodiments. In some embodiments, a silk material may be or comprise silk fibroin (e.g., degummed or substantially sericin free silk fibroin). In some embodiments, a silk material may be or comprise silk powder (e.g., comprising a plurality of silk particles).

In some embodiments, a silk fibroin material may be or comprise silk particles (e.g., microparticles or nanoparticles). As used herein, the term “particles” includes spheres, rods, shells, prisms, and related structures. While any application-appropriate particle size is contemplated as within the scope of the present disclosure, in some embodiments, a silk particle be have a diameter between 1 nm and 1,000 μm (e.g., between 1 nm and 1 μm, between 1 μm and 1,000 μm, etc). In some embodiments, a silk particle may have a diameter of greater than 1,000 μm.

Various methods of producing silk particles (e.g., nanoparticles and microparticles) are known in the art. For example, a milling machine (e.g., a Retsch planetary ball mill) can be used to produce silk powder. Generally, the ball mill consists of either two or four sample cups arranged around a central axis, which is geared such that each cup rotates both centrally and locally. Each ceramic cup is filled with small ceramic spheres. A range of sizes is available; balls with a diameter of 10 millimeters were/are used for the milling operations described in the present disclosure. As the cups spin, the spheres crush material in the cups to a small characteristic size. Both degummed and non-degummed silk can be converted from pulverized material to powder form in the ball mill.

In other embodiments, alternative powder formation techniques can be used (e.g., lyophilization or flash freezing and crushing). In other embodiments, alternative grates on the pulverizer, with larger holes, can be used. This can generate larger silk particle sizes.

In some embodiments, silk particles can be produced using a freeze-drying method as described in U.S. Provisional Application Ser. No. 61/719,146, filed Oct. 26, 2012, content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some embodiments, the silk fibroin material comprising substantial amounts of amorphous structure is prepared from silk solution and is composed of nanostructures, an may be referred to as nano-sized silk powder (NSP) and be part of materials referred to amorphous silk nanomaterials (ASN). As used herein, these terms are equivalent and may be used interchangeably.

Without wishing to be held to a particular theory, in some embodiments, the present disclosure encompasses the recognition that the use of particular starting materials (e.g., silk fibroin material comprising substantial amounts of amorphous structure) allows for the production of previously unattainable compositions. In some embodiments, a silk material is not made from solubilized silk. In some embodiments, a silk material may be lyophilized.

Silk Fibroin

According to various embodiments, any silk fibroin may be used in provided methods. In some embodiments, the silk fibroin is selected from the group consisting of spider silk (e.g., from Nephila ciavipes), silkworm silk (e.g., from Bombyx mori), and recombinant silks (e.g., produced/engineered from bacterial cells, yeast cells, mammalian cells, transgenic animals, and/or transgenic plants). In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected, is present in an amount that is below detection, or is absent.

If necessary, a silk solution may be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, silk fibroin and/or a silk fibroin article core, may comprise a protein structure that substantially includes β-turn and/or β-strand regions. Without wishing to be bound by a theory, the silk R sheet content can impact gel function and in vivo longevity of the composition. It is to be understood that composition including non-β sheet content (e.g., e-gels) can also be utilized. In some embodiments, silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk R sheet content, from less than 10% to ˜ 55% can be used in the silk fibroin compositions disclosed herein.

In some embodiments, silk fibroin, or a silk fibroin article core, has a protein structure that is substantially-free of α-helix and/or random coil regions. In aspects of these embodiments, the silk fibroin has a protein structure including, e.g., about 5% α-helix and/or random coil regions, about 10% α-helix and/or random coil regions, about 15% α-helix and/or random coil regions, about 20% α-helix and/or random coil regions, about 25% α-helix and/or random coil regions, about 30% α-helix and/or random coil regions, about 35% α-helix and/or random coil regions, about 40% α-helix and/or random coil regions, about 45% α-helix and/or random coil regions, or about 50% α-helix and/or random coil regions. In other aspects of these embodiments, the silk fibroin has a protein structure including, e.g., at most 5% α-helix and/or random coil regions, at most 10% α-helix and/or random coil regions, at most 15% α-helix and/or random coil regions, at most 20% α-helix and/or random coil regions, at most 25% α-helix and/or random coil regions, at most 30% α-helix and/or random coil regions, at most 35% α-helix and/or random coil regions, at most 40% α-helix and/or random coil regions, at most 45% α-helix and/or random coil regions, or at most 50% α-helix and/or random coil regions. In yet other aspects of these embodiments, the silk fibroin has a protein structure including, e.g., about 5% to about 10% α-helix and/or random coil regions, about 5% to about 15% α-helix and/or random coil regions, about 5% to about 20% α-helix and/or random coil regions, about 5% to about 25% α-helix and/or random coil regions, about 5% to about 30% α-helix and/or random coil regions, about 5% to about 40% α-helix and/or random coil regions, about 5% to about 50% α-helix and/or random coil regions, about 10% to about 20% α-helix and/or random coil regions, about 10% to about 30% α-helix and/or random coil regions, about 15% to about 25% α-helix and/or random coil regions, about 15% to about 30% α-helix and/or random coil regions, or about 15% to about 35% α-helix and/or random coil regions.

Elevated Temperatures

As discussed herein, provided methods and compositions include the exposure to elevated temperature(s). As used herein, the term “elevated temperatures” refers to temperatures higher than standard room temperature (i.e., greater than 25° C.). In some embodiments, provided methods or compositions include exposure to a single elevated temperature. In some embodiments, provided methods or compositions include exposure to at least two elevated temperatures (e.g., 3, 4, 5, 6, 7, 8, 9, 10 or more). In some embodiments where a method of composition includes two or more elevated temperatures, at least two of those elevated temperatures are different from one another.

In some embodiments, an elevated temperature may be between 25° C. and 200° C. By way of specific exemplary ranges, in some embodiments, an elevated temperature may be between 25° C. and 150° C., between 25° C. and 100° C., between 25° C. and 95° C., between 25° C. and 50° C., between 50° C. and 200° C., between 50° C. and 150° C., between 50° C. and 100° C., between 25° C. and 100° C. between 125° C. and 200° C., or any other range between 125° C. and 175° C.

In some embodiments, an elevated temperature may be at least 25° C. By way of additional example, in some embodiments, an elevated temperature may be at least 26° C., 27° C., 28° C., 29° C., 30° C., 35° C., 40° C., 45° C., 50° C., 55° C., 60° C., 65° C., 70° C., 75° C., 80° C., 85° C., 90° C., 95° C. or 100° C. In some embodiments, enhanced crystallization of silk fibroin material is observed at temperatures at or above 95° C.

In some embodiments, an elevated temperature may be at most 125° C. By way of additional example, in some embodiments, an elevated temperature may be at most 126° C., 127° C., 128° C., 129° C., 130° C., 135° C., 140° C., 145° C., 150° C., 155° C., 160° C., 165° C., 170° C., 175° C., 180° C., 185° C., 190° C., or 195° C.

Application of elevated temperature(s) to a provided composition or in a provided method may occur in any application-appropriate manner. By way of non-limiting example, in some embodiments, application of elevated temperature(s) may be via heat pressing, via a heating device such as an oven, heating stage, exposed flame or other mechanism.

Application of elevated temperature(s) may occur at or over any of a variety of time periods. For example, in some embodiments, application of elevated temperature(s) occurs substantially instantly (e.g., by placement over a flame or in an oven). In some embodiments, application of elevated temperature(s) occurs over a period of seconds, minutes, or hours. In some embodiments, application of elevated temperature(s) occurs over a period of time between 1 second and 1 hour.

Elevated Pressure

As discussed herein, provided methods and compositions include the exposure to elevated pressure(s). As used herein, the term “elevated pressures” refers to pressures higher than standard atmospheric pressure (i.e., 1.013 bar). In some embodiments, provided methods or compositions include exposure to a single elevated pressure. In some embodiments, provided methods or compositions include exposure to at least two elevated pressures (e.g., 3, 4, 5, 6, 7, 8, 9, 10 or more). In some embodiments where a method of composition includes two or more elevated pressures, at least two of those elevated pressures are different from one another.

Any application-appropriate method(s) may be used to cause elevated pressure as applied to provided compositions or in provided methods. By way of non-limiting example, in some embodiments, elevated pressure may include use of a vacuum, a press (e.g. heat press), and combinations thereof.

In some embodiments, application of elevated pressure may be or include uniaxial compression. In some embodiments, application of elevated pressure may be or include multi-axial compression (e.g., biaxial compression).

While any application-appropriate level of elevated pressure may be used, in some embodiments, an elevated pressure between 1 MPa and 1 GPa is used. By way of specific exemplary ranges, in some embodiments, an elevated pressure may be between 10 MPa and 1 GPa, between 50 MPa and 1 GPa, between 100 MPa and 1 GPa, between 200 MPa and 1 GPa, between 300 MPa and 1 GP, between 400 MPa and 1 GPa or between 500 MPa and 1 GPa. In some embodiments, an elevated pressure may be or comprise at least 1 MPa (e.g., at least 2 MPa, 3 MPa, 4 MPa, 5 MPa, 6 MPa, 7 MPa, 8 MPa, 9 MPa, 10 MPa, 20 MPa, 30 MPa, 40 MPa, 50 MPa, 60 MPa, 70 MPa, 80 MPa, 90 MPa, 100 MPa 150 MPa, 200 MPa, 250 MPa, 300 MPa, 350 MPa, 400 MPa, 450 MPa, 500 MPa, 550 MPa, 600 MPa, 650 MPa, 700 MPa, or 750 MPa).

Silk Article Cores

In some embodiments, provided silk article cores exhibit a substantially homogenous structure. As used herein, “substantially homogenous structure” means that silk fibroin molecules are distributed and/or configured in a consistent way throughout substantially all of a portion of or the entirety of an article. Further, in some embodiments, silk article cores may exhibit significant amounts of silk fibroin in a semi-crystalline structure. In some embodiments, production of a silk article core according to provided methods includes a transition on the structure of silk fibroin from a substantially amorphous state to a semi-crystalline state, for example, as observed via X-ray diffraction.

In some embodiments, a silk article core may exhibit significant amounts of 3-sheet structure. For example, in some embodiments, a silk article core may exhibit at least 10 wt % more (e.g., at least 20 wt %, 30 wt %, 40 wt %) j-sheet structures as compared to the starting silk fibroin material. In some embodiments, a silk article core may exhibit at least 50 wt % more (e.g., at least 60 wt %, 70 wt %, 80 wt %, 90 wt %, 95 wt %) β-sheet structures as compared to the starting silk fibroin material.

In some embodiments, crystallinity of silk article cores may be controlled by the application of temperature and pressure. For example, in some embodiments, when amorphous silk is processed at temperatures ranging from about 25° C.-125° C., the silk article core may contain about 10-15% β-sheet structures. In some embodiments when amorphous silk is processed at temperatures ranging from about 125° C.-175° C., the silk article core may contain for example, about 20-35% β-sheet structures or for example, over 40% β-sheet structures.

In some embodiments, provided methods and compositions allow for the production of silk article cores which that are homogenous, where the silk amorphous powders are packed together via the bonding between neighboring raw silk powders, for example, at processing temperatures of about 25° C.-95° C. In some embodiments, provided methods and compositions allow for the production of silk article cores which that are homogenous, where the silk molecules of amorphous powders gain more mobility as they are heated above the glass transition temperature and self-assemble into interlocked nanoglobules, for example, at processing temperatures of about 125° C.-175° C.

In some embodiments, provided methods and compositions allow for the production of silk article cores (e.g., thin films) that undergo thermal softening and are bendable and moldable into a desired shape. In some embodiments, provided methods and compositions allow for the production of silk article cores that are machinable.

Provided methods and compositions allow for the production of complex silk article cores in ways that were not achievable using previous methods (e.g., silk screws that can resist torsion forces relevant to in vivo use). By way of non-limiting example, in some embodiments provided methods and compositions may be used to produce silk article cores such as films, fibers, meshes, needles, tubes, plates, screws, rods, and any combination thereof.

In some embodiments, a silk article core may be amenable to one or more types of patterning. In some embodiments, patterning may be or comprise macropatterning. In some embodiments, patterning may be or comprise micropatterning (i.e., patterning with micro scale features). In some embodiments, patterning may be or comprise nanopatterning (i.e., patterning with nano scale features). In some embodiments, patterning may be or comprise: etching, lithography-based patterning, carving, cutting, and any combination thereof.

In some embodiments, a silk article core may be subjected to one or more types of processing (e.g., 2, 3, 4, 5, 6, 7, 8, 9, 10 or more). While any application-appropriate form of processing is contemplated as within the scope of the present disclosure, in some embodiments, processing may be or comprise machining, rolling, drilling, milling, sanding, punching die cutting, extruding, chemical etching, coating, molding, turning, thread rolling, and any combination thereof.

Exemplary Properties or Characteristics of Silk Article Cores

In some embodiments, provided compositions (e.g., silk article cores) may be substantially transparent. In some embodiments, provided compositions (e.g., silk article cores) may be semi-transparent. In some embodiments, provided compositions (e.g., silk article cores) may be substantially non-transparent. As used herein, the term “transparent” refers to the propensity of an object to transmit light (with or without scattering of said light). In some embodiments, a composition/article is said to be substantially transparent if it transmits ≥80% of light it is exposed to in the visible range (400 nm-800 nm). In some embodiments, a composition/article is said to be semi-transparent if it transmits between 50%-80% of light it is exposed to in the visible range (400 nm-800 nm). In some embodiments, a composition/article is said to be substantially non-transparent if it transmits ≤50% of light it is exposed to in the visible range (400 nm-800 nm).

In some embodiments, provided compositions may be biocompatible and/or biodegradable. In some embodiments, provided compositions may exhibit particular degradation profile(s). By way of specific example, in some embodiments, a provided composition may degrade at least 50 wt % after about 96 hours of exposure to an aqueous environment at 37° C. In some embodiments, a provided composition may not degrade more than 10% after months of exposure to an in vivo environment or condition.

In some embodiments, provided compositions may exhibit one or more desirable properties including, but not limited to: electrical conductivity, enhanced machinability, and/or enhanced thermoformability.

Additives

In some embodiments, provided methods and compositions include one or more additives (e.g., 2, 3, 4, 5, 6, 7, 8, 9, 10 or more). In some embodiments, at least one additive may be mixed with or otherwise associated with a silk fibroin material prior to an applying step (e.g. exposure to one or more of elevated temperature and elevated pressure). In some embodiments, at least one additive may be mixed with or otherwise associated with a silk fibroin material substantially at the same time as an applying step). In some embodiments, at least one additive may be mixed with or otherwise associated with a silk fibroin material subsequently to an applying step.

Provided methods and compositions are amenable to the addition of any of a variety of additives. By way of non-limiting example, in some embodiments an additive may be or comprise a small molecule, an organic macromolecule, an inorganic macromolecule, an electrically conductive material, an inorganic material, a hydrophobic material, a hydrophilic material, a nanomaterial, and any combination thereof.

The processing of the silk-based materials, including pure silk materials and silk-based composite materials, can be modified with addition of one or more additives. In some embodiments, a function of an additive may be to tune the processing conditions and the properties of the products. In some embodiments, additives may be selected from water; glycerol; saccharides; biological macromolecules, e.g. peptide, proteins; antibodies and antigen binding fragments; nucleic acids; immunogens; antigens; enzyme; synthetic polymers, e. g. poly(ethylene) glycol, poly-lactic acid, poly(lactic-co-glycolic acid) to name but a few specific examples, though any application-appropriate additive is specifically contemplated as within the scope of the present disclosure.

In some embodiments, for example some embodiments contemplated for in vivo use, provided compositions may comprise one or more proteases. In some embodiments, an organic macromolecule is or comprises at least one protease. In some embodiments, a protease is or comprises one or more of Proteinase XIV, Proteinase K, a-chymotrypsin, collagenase, matrix metalloproteinase-1 (MMP-1), and MMP-2. In some embodiments, a protease may be useful in tailoring the degradation profile of a particular provided composition (e.g., in an in vivo environment).

In some embodiments, an electrically conductive material may be or comprise an organic conductive material and/or an inorganic conductive material (e.g., a metal). In some embodiments, an electrically conductive material may be or comprise at least one of a conductive polymer, graphene, silver, gold, aluminum, copper, platinum, steel, brass, bronze, and iron oxide.

Any application-appropriate amount of one or more additives may be useful according to various embodiments. By way of non-limiting example, in some embodiments, an additive may be present in a provided composition in an amount between 0.001 wt % and 95 wt %. In some embodiments, one or more additives may be mixed with a silk fibroin material in an amount ranging between 0.001 wt % and 95 wt % of the silk fibroin material.

### EXAMPLES

### Example 1

Materials: Poly lactic acid filament for 3D printing, silk bars made from thermal molding process (95° C., 625 MPa).

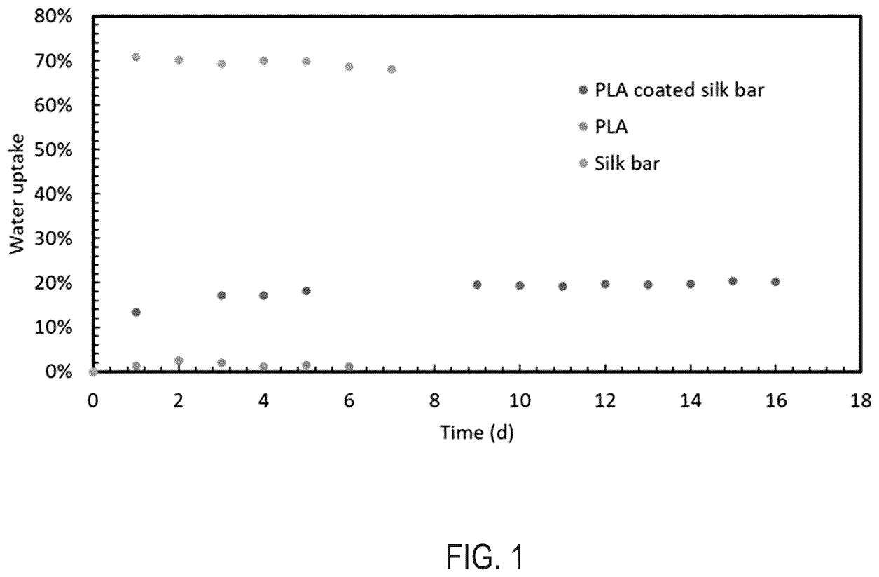

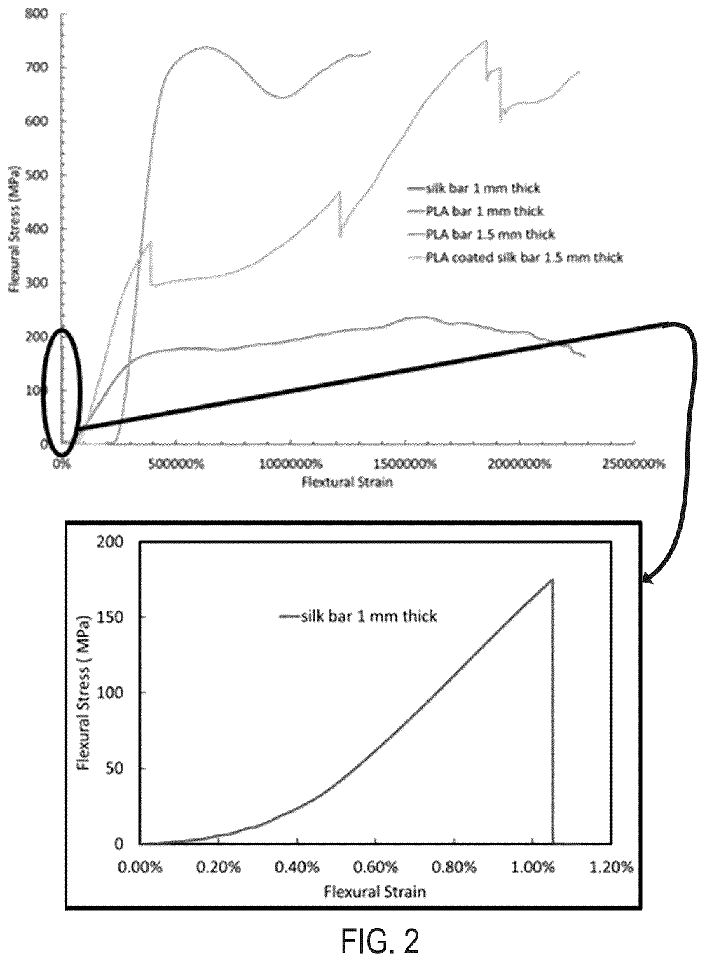

Methods: 3D printing was used to coat the silk articles with PLA, using the fused deposition modeling (FDM) approach on a Flashforge CreatorPro 3D printer. The geometry of the PLA shell was designed using CAD (Solidworks) with respect to the dimensions of the silk articles. The design of the shell was arranged in two parts: a shell body and a cap. The body of the shell was printed first; the silk articles were then placed inside the shell, with subsequent printing of the cap of the shell. The PLA-coated silk articles were immersed in PBS solution at 37° C. The PLA articles were also 3D printed with the same size as the silk articles (32 mm×1 mm×6.5 mm). The weight of PLA coated silk articles, uncoated silk articles, and PLA articles were measured before immersion in PBS solution, and after immersion in PBS solution overnight. FIG. 1 is a plot showing the water uptake for these three materials. FIG. 2 is a plot showing measured flexural strain for samples of these materials.

### Comparative Example 1

Materials: Beeswax (Millipore-Sigma, St. Louis, MO), silk fibroin articles made from thermal molding method (95° C., 625 MPa), silk fibroin amorphous nanoparticles.

Methods: a certain amount of beeswax was heated until melted in a proper sized beaker. The silk articles were immersed in the melted beeswax for dip coating. The coating was repeated two times and the articles were cooled until a fine layer of wax was coated on the silk article. The wax coated silk articles were immersed in PBS solution at 37° C. The weight of the wax coated silk articles was measured before immersion in PBS solution, and after immersion in PBS solution overnight. Silk-beeswax composite articles were prepared by first mixing beeswax powder (10%) with silk amorphous nanoparticles via analytical milling (20,000 r.p.m., 2 min, Col-Parmer), then the mixed powder was thermal molded under 625 MPa, 95° C. for 15 minutes. The obtained articles were immersed in PBS solution at 37° C. The weight of the wax combined silk articles was measured before immersion in PBS solution, and after immersion in PBS solution overnight. The wax coated silk articles had greater water uptake than the PLA-coated silk articles from Example 1. The wax combined silk articles decreased water uptake from 34% to 27%, which is less reduction in water uptake than the PLA-coated silk articles from Example 1.

Equivalents and Scope. The recitation of a listing of elements in any definition of a variable herein includes definitions of that variable as any single element or combinations (or sub-combinations) of listed elements. The recitation of an embodiment herein includes that embodiment as any single embodiment or in combination with any other embodiments or portions thereof. Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A coated silk fibroin article having reduced water uptake and/or improved mechanical properties, the coated silk fibroin article comprising:
a silk fibroin article core; and
a biodegradable hydrophobic polymer layer substantially encompassing the silk fibroin article core,
wherein the coated silk fibroin article has either: (i) at least one mechanical property that is at least 50% greater than a reference mechanical property of the silk fibroin article core in an absence of the biodegradable hydrophobic polymer layer, wherein the at least one mechanical property includes a three-point bending flexural strain; or (ii) a water uptake that is at least 50% lower than a reference water uptake of the silk fibroin article core in the absence of the biodegradable hydrophobic polymer layer.

2. The coated silk fibroin article of claim 1, wherein the silk fibroin article core is a thermally-processed silk fibroin article core.

3. (canceled)

4. A method comprising:
(i) providing a silk fibroin material comprising substantially amorphous structure;
(ii) applying at least one of elevated temperature and elevated pressure to the silk fibroin material to form a silk fibroin article core, wherein the applying induces fusion between at least a portion of the silk fibroin material and structural change of fibroin in the silk fibroin material; and
(iii) coating the silk fibroin article core with a biodegradable hydrophobic polymer layer to form a coated silk fibroin article.

5.-7. (canceled)

8. The coated silk fibroin article of claim 1, the biodegradable hydrophobic polymer layer comprising aliphatic polyesters selected from the group consisting of polylactide, polycaprolactone, and polycarbonate, aromatic and aliphatic polyanhydrides, polyurethanes, polyamides, poly(ester amide), or a copolymer or combination thereof.

9. The coated silk fibroin article of claim 1, the biodegradable hydrophobic polymer layer comprising poly(caprolactone).

10. The coated silk fibroin article of claim 1, the biodegradable hydrophobic polymer layer having a substantially uniform thickness.

11. The coated silk fibroin article of claim 1, the biodegradable hydrophobic polymer layer having an average thickness of between 1 μm and 5 mm.

12. The coated silk fibroin article of claim 1, wherein the biodegradable hydrophobic polymer layer accounts for no more than 50% by weight of the coated silk fibroin article and at least 1% by weight of the coated silk fibroin article.

13. The coated silk fibroin article of claim 1, the silk fibroin article core and/or the biodegradable hydrophobic polymer layer comprising an acid-activated protease.

14. The coated silk fibroin article of claim 13, wherein the acid-activated protease is selected from the group consisting of pepsin, a cathepsin, renin, and combinations thereof.

15. The coated silk fibroin article of claim 13, wherein the acid-activated protease is triggered by a local pH reduction initiated by dissolution or degradation of the biodegradable hydrophobic polymer layer.

16. The coated silk fibroin article of claim 1, the coated silk fibroin article having a flexural strength of at least 50 MPa, at least 100 MPa, or at least 150 MPa.

17. The coated silk fibroin article of claim 1, the coated silk fibroin article and/or the silk fibroin article core having a density of at least 1.20 g/cm³.

18. The method of claim 4, wherein the applying comprises application of both elevated temperature and elevated pressure to the silk fibroin material.

19. The coated silk fibroin article of claim 1, wherein the silk fibroin article core is substantially homogeneous.

20. The coated silk fibroin article of claim 1, wherein the silk fibroin article core comprises silk in an amount of about 10% (w/w) or higher.

21.-46. (canceled)

47. The coated silk fibroin article of claim 1, wherein the coated silk fibroin article is at least a portion of a packaging material.

48-50. (canceled)

51. The coated silk fibroin article of claim 1, wherein the coated silk fibroin article has the at least one mechanical property that is at least 50% greater than a reference mechanical property of the silk fibroin article core in the absence of the biodegradable hydrophobic polymer layer.

52. The coated silk fibroin article of claim 1, wherein the water uptake is at least 50% lower than a reference water uptake of the silk fibroin article core in the absence of the biodegradable hydrophobic polymer layer.
